(19) United States
(12) Patent Application Publication
(10) Pub. No.: US 2013/0017278 A1
(43) Pub. Date: Jan. 17, 2013

(54) COMPOSITION AND METHOD FOR IMPROVING BRAIN FUNCTION

(71) Applicant: Arlene Keller, Calgary (CA)

(72) Inventor: Arlene Keller, Calgary (CA)

(21) Appl. No.: 13/547,661

(22) Filed: Jul. 12, 2012

Related U.S. Application Data
(60) Provisional application No. 61/457,940, filed on Jul. 13, 2011.

(51) Int. Cl. A61K 36/889; A61P 25/00

(52) U.S. Cl. 424/727

(57) ABSTRACT

A composition and method for improving and maintaining brain function wherein the composition comprises an effective amount of coconut oil and an effective amount of medium-chain triglycerides. The medium-chain triglycerides and coconut oil may be mixed in a ratio of between 90% medium-chain triglycerides to 10% coconut oil and 40% medium-chain triglycerides to 60% coconut oil. The method comprises administering to a person, an effective amount of coconut oil and an effective amount of medium-chain triglycerides. The composition may be administered in a food item.

### CROSS REFERENCE TO RELATED APPLICATION

This application claims priority from U.S. Provisional Patent Application No. 61/457,940 filed Jul. 13, 2011 entitled Composition and Method for Improving Brain Function.

### BACKGROUND OF THE INVENTION

1. Field of Invention

The present invention relates to the field of neurological functioning in general and in particular to a method of treating Alzheimer's Disease, and other disorders affecting neurological function.

2. Description of Related Art

Many types of diseases and degenerations affect mental or neurological functioning. Examples of such cognitive disorders may include Alzheimer's disease, Parkinson's disease and elderly onset dementia. Although many types of cognitive disorders affect primarily the elderly, people of all ages may be affected. Common effects in a person afflicted by a cognitive disorder include memory difficulties, reduced ability to learn, reason, retain or recall past experience as well as loss of patterns of thoughts, feelings and activities confusion, irritability and aggression, mood swings, language breakdown, long-term memory loss, and the general withdrawal of the sufferer as their senses decline. Additional difficulties may often affect people with cognitive disorders including reduced quality of life, including the need for caregivers and/or institutionalization. Individuals with some types of cognitive disorders may also neglect themselves and may become uninhibited as their condition worsens.

Many of the current treatments have not been effective in controlling or preventing the advancement of the cognitive disorder. Previous approaches to treatment of many types of cognitive disorders have been primarily directed towards treating the symptoms associated with cognitive disorders. Examples of such treatments for symptoms may include pain, anxiety, antidepressant or antipsychotic medications, many of which have undesirable side effects.

### SUMMARY OF THE INVENTION

According to a first embodiment of the present invention there is disclosed a method for improving and maintaining brain function comprising administering to a person, an effective amount of coconut oil and an effective amount of medium-chain triglycerides.

The coconut oil may comprise extra virgin coconut oil. The coconut oil may be administered orally. The coconut oil may be administered in a food item. The coconut oil may be administered in a plurality of doses within a 24 hour period. The coconut oil may be administered in at least 4 doses within a 24 hour period.

The medium-chain triglycerides may be administered in at least two stages. The medium-chain triglycerides may be administered in a first stage. The first stage may comprise administering 2 tablespoons of medium-chain triglycerides divided evenly among 3 meals and one snack daily. The first stage may be approximately one week in length. The medium-chain triglycerides may be administered in a second stage. The second stage may comprise administering 4 tablespoons or 10 teaspoons of medium-chain triglycerides divided evenly among 3 meals and one snack daily. The second stage may have an indefinite duration.

The medium-chain triglycerides may be administered orally. The medium-chain triglycerides may be administered in a food item. The medium-chain triglycerides may be administered in a plurality of doses within a 24 hour period. The medium-chain triglycerides may be administered in at least 4 doses within a 24 hour period.

The medium-chain triglycerides and coconut oil may be mixed in a single composition. The composition may be administered orally. The composition may be administered in a food item.

The composition may be administered in a plurality of doses within a 24 hour period. The composition may be administered in at least 4 doses within a 24 hour period. The composition may be administered in at least two stages. The composition may be administered in a first stage comprising administering 2 tablespoons of medium-chain triglycerides divided evenly among 3 meals and one snack daily. The first stage may be approximately one week in length.

The composition may be administered in a second stage comprising administering 4 tablespoons or 10 teaspoons of medium-chain triglycerides divided evenly among 3 meals and one snack daily. The second stage may have an indefinite duration.

According to a first embodiment of the present invention there is disclosed a composition for improving and maintaining brain function comprising an effective amount of coconut oil and an effective amount of medium-chain triglycerides. The medium-chain triglycerides and coconut oil may be mixed in a ratio of between 90% medium-chain triglycerides to 10% coconut oil and 40% medium-chain triglycerides to 60% coconut oil. The medium-chain triglycerides and coconut oil may be mixed in a ratio of 4 to 1. The medium-chain triglycerides and coconut oil are mixed in a ratio of 2 to 3.

Other aspects and features of the present invention will become apparent to those ordinarily skilled in the art upon review of the following description of specific embodiments of the invention in conjunction with the accompanying description.

### DETAILED DESCRIPTION

It is the novel insight of this invention that medium chain triglycerides (MCT) in conjunction with coconut oil are useful as a treatment and preventative measure for cognitive disorders, such as by way of non-limiting example, mild cognitive impairment, dementia and related illnesses such as Alzheimer's disease, Dementia with Lewy Bodies, Huntington's disease, Parkinson's disease, and the like.

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein. As used herein, “patient” refers to any mammal, including humans that may benefit from treatment of disease and conditions resulting from reduced neuronal metabolism.

The present invention provides a method of treating cognitive disorders by administering to a patient, an effective therapeutic amount of medium- chain triglycerides along with an effective therapeutic amount of coconut oil. In some embodiments of the present invention, the MCT and coconut oil may be administered to the patient in phases so as to increase the amount of MCT oil that is administered to the patient.

Medium chain triglycerides, (MCT) are composed of fatty acid esters of glycerol with chain lengths of between 5-12 carbons. Sources of common MCTs may include coconut oil, palm kernel oils and camphor tree drupes such as provided by Nature's Blessings of the Philippines. Coconut oil may advantageously be virgin coconut oil and may also advantageously be organic virgin coconut oil such as is provided from Jedwards International Inc. of Quincy, Mass.

“Effective amount” refers to an amount of a compound, material, or composition, as described herein that is effective to achieve a particular biological result. Such results include, but are not limited to, at least one of the following: enhancing cognitive function, improving memory, improving liver function, increasing daytime activity, improving learning, improving attention, improving social behavior, improving motor performance, and/or improving cerebrovascular function, particularly in aging or geriatric mammals. In various embodiments, “effective amount” refers to an amount suitable to reverse, reduce, prevent, or delay a decline in the above qualities, for example, cognitive function or performance, memory, learning rate or ability, problem solving ability, attention span and ability to focus on a task or problem, motor function or performance, social behavior, and the like.

The term “cognitive function” refers to the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness. “Enhanced cognitive function” or “improved cognitive function” refers to any improvement in the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness, as measured by any means suitable in the art.

The present invention relates to any animal, preferably a mammal, and more preferably, humans. The compositions and methods provided herein and below are generally intended for long term consumption which may be considered to be periods of longer than 2 months which may be considered to be indefinite as having no set end period. In some situations, it is envisioned that the patient would continue to consume the compositions according to the present methods for the remainder of its life.

In some embodiments of the present invention, the MCT oil and coconut oil may be administered to the patient orally. As used herein, the term “oral administration” or “orally administering” means that the patient ingests, or a caretaker is directed to feed, or does feed, the patient one or more of the compositions described herein. In some embodiments, the MCT oil and coconut oil may be incorporated into or mixed within the food of the patient for consumption at regular intervals at meal and snack times. In particular, the MCT oil and coconut oil may be incorporated into 3 meals and at least one snack daily of the patient. Optionally, the MCT oil and coconut oil may be orally provided to the patient in any known method, such as by way of non-limiting example, as a liquid or capsule or incorporated into tablets, lozenges, troches, hard candies, nutritional bars, nutritional drinks, metered sprays, creams, and suppositories, among others. The compositions may be combined with a pharmaceutically acceptable excipient such as gelatin, an oil, and/or other pharmaceutically active agent(s). Optionally, the MCT oil and coconut oil may be utilized as a cooking oil or salad dressing as a method of administering the compositions to the patient.

In a preferred embodiment, the MCT oil and coconut oil may be administered to the patient in an increasing amount from an initial or base amount of a coconut oil, and thereafter, an amount of MCT oil may be administered to the patient in an increasing amount until the indefinite dosage is reached. Thereafter both the MCT oil and coconut oil may be administered to the patient indefinitely.

In an alternative embodiment, the MCT oil and coconut oil may be mixed together within a single composition according to known methods. It will be appreciated that the MCT oil and coconut oil may be mixed together in any known ratio, such as, by way of non-limiting example a ratio of between 90% MCT oil to 10% coconut oil to 40% MCT oil with 60% coconut oil. In such an embodiment, the composition containing the MCT oil and coconut oil may be administered to the patent in an increasing amount so to reduce any side effects associated with the MCT oil and coconut oil. By way of non-limiting example, the composition may be administered to the patient in an amount of one teaspoon at each of 3 meals and at least one snack daily of the patient for the first week. Thereafter, and continuing indefinitely, the composition may be administered to the patient in an amount of 2.5 teaspoons at each of 3 meals and 2 to 3 snacks daily. It will be appreciated that the amount of composition administered to the patient may be varied from this amount depending upon the needs and tolerance of the patient. According to a further embodiment of the present invention, the above composition may also be useful as a composition for use in assisting weight loss. Additionally, according to further embodiments of the present invention, the above compositions may also be useful as a cooking oil or salad dressing as a method of administering the compositions to the patient.

### EXAMPLES

The following examples are offered by way of illustration and not by way of limitation.

### Example 1

During the first month of treatment, a patient was administered 4 tablespoons (59 ml) of coconut oil divided substantially equally into 3 meals and one snack (or at 4 food consumptions) per day. This coconut oil dosage is continued indefinitely. During the second month of treatment, the patient is administered an increasing amount of MCT oil in addition to the coconut oil. During the first week of the second month, the quantity of MCT oil administered to the patient is 1 teaspoon (5 ml) divided equally into 3 meals per day. During the second week of the second month, the quantity of MCT oil administered to the patient is 2 teaspoon (10 ml) divided equally into 3 meals per day. During the third week of the second month, the quantity of MCT oil administered to the patient is 3 teaspoon (15 ml) divided equally into 3 meals per day. During the fourth week of the second month and for an indefinite amount of time thereafter), the quantity of MCT oil administered to the patient is 4 teaspoon (20 ml) divided equally into 3 meals and one snack (or at 4 food consumptions) per day.

Using the above method, the patient began the method and had scores of between 17 and 18 on the Alzheimer's Mini-Mental State Exam. After one year of continuously being administered MCT oil and coconut oil according to the above method, the patient had scores of 21 on the Alzheimer's Mini-Mental State Exam.

While specific embodiments of the invention have been described and illustrated, such embodiments should be considered illustrative of the invention only and not as limiting the invention as construed in accordance with the accompanying claims.

What is claimed is:

1. A method for improving and maintaining brain function comprising administering to a person, an effective amount of coconut oil and an effective amount of medium-chain triglycerides.

2. The method of claim 1 wherein said coconut oil comprises extra virgin coconut oil.

3. The method of claim 1 wherein said medium-chain triglycerides and coconut oil are mixed in a single composition.

4. The method of claim 3 wherein said composition is administered orally.

5. The method of claim 4 wherein said composition is administered in a food item.

6. The method of claim 3 wherein said composition is administered in a plurality of doses within a 24 hour period.

6. The method of claim 6 wherein said composition is administered in at least 4 doses within a 24 hour period.

7. The method of claim 3 wherein said composition is administered in at least two stages.

8. The method of claim 7 wherein said composition is administered in a first stage comprising administering 2 tablespoons of medium-chain triglycerides divided evenly among 3 meals and one snack daily.

9. The method of claim 8 wherein said first stage is approximately one week in length.

10. The method of claim 7 wherein said composition is administered in a second stage comprising administering 4 tablespoons or 10 teaspoons of medium-chain triglycerides divided evenly among 3 meals and one snack daily.

11. The method of claim 10 wherein said second stage has an indefinite duration.

12. A composition for improving and maintaining brain function comprising an effective amount of coconut oil and an effective amount of medium-chain triglycerides.

13. The composition of claim 1 wherein said medium-chain triglycerides and coconut oil are mixed in a ratio of between 90% medium-chain triglycerides to 10% coconut oil and 40% medium-chain triglycerides to 60% coconut oil.

14. The method of claim 13 wherein said medium-chain triglycerides and coconut oil are mixed in a ratio of 4 to 1.

15. The method of claim 13 wherein said medium-chain triglycerides and coconut oil are mixed in a ratio of 2 to 3.
